(19) United States
(12) Patent Application Publication
(10) Pub. No.: US 2003/0207310 A1
(43) Pub. Date: Nov. 6, 2003

(54) System and method for determining the sizes and quantity of polynucleotides with capillary array electrophoresis

(76) Inventors: Zhaowei Liu, Port Matilda, PA (US); Zhiyong Guo, State College, PA (US); Qingbo Li, State College, PA (US); Thomas E. Kane, State College, PA (US)

Correspondence Address: PENNIE & EDMONDS LLP, 1667 K STREET NW, SUITE 1000, WASHINGTON, DC, 20006

(21) Appl. No.: 10/425,746

(22) Filed: Apr. 30, 2003

Related U.S. Application Data
(60) Provisional application No. 60/376,565, filed on May 1, 2002.

(51) Int. Cl. C12Q 1/68; G06F 19/00; G01N 33/48; G01N 33/50; G01L 1/20

(52) U.S. Cl. 435/6; 204/451; 702/20

(57) ABSTRACT

An electrophoretic separation system configured to determine a size of each of a plurality of sample polynucleotides includes a plurality of sample separation lanes, such as capillaries. Each separation lane is configured to subject a number plurality of respective sample polynucleotides and a respective internal standard polynucleotide (ISP) to electrophoresis. The system also includes a ladder separation lane for subjecting at least two members of polynucleotide ladder to electrophoresis. A processor of the system is configured to determine migration coordinates of (1) the ISP and sample polynucleotides subjected to electrophoresis within each separation lane and (2) at least two of the PLMs. The processor also transforms the migration coordinates of the sample polynucleotides of each separation lane from a migration dimension of their respective separation lane to a migration dimension of the polynucleotide ladder. The sizes of the sample polynucleotides are determined based on (1) the respective transformed migration coordinates thereof and (2) migration coordinates of at least two PLMs.

### RELATED APPLICATIONS

This application claims the benefit of provisional application No. 60/376,565, filed May 1, 2002, which application is incorporated herein.

### FIELD OF THE INVENTION

The present invention relates to systems and methods for obtaining electrophoresis data indicative of the size and quantity of polynucleotides present in a sample.

### BACKGROUND OF THE INVENTION

Polymerase chain reaction (PCR) has become an essential tool for genetic analysis. Many high-throughput applications including DNA sequencing, transcription expression profiling, mutation detection, genome mapping, cloning analysis and diagnosis of clinical samples require large sets of amplified DNA products as starting material. The PCR process has also been widely used in testing for the presence of genetically modified organisms (GMOs). PCR products, however, must be analyzed, such as to determine their size and quantity, before they are used in further applications. Thus, there is a high demand for the development of a high-throughput method capable of rapidly assessing the size and quantity of PCR products. The most common method of assessment involves agarose gel electrophoresis and subsequently densitometric scanning to determine the size and concentration of the product. The procedure is time-consuming, labor-intensive and consumes large volume of buffer even when precast gel systems are used. In addition, the estimation of the PCR fragment size and quantity is often unsatisfactory. Alternatively, the analysis of PCR amplicons subjected to electrophoresis in the presence of a molecular ladder has been performed using single electrophoresis capillaries.

A method for identifying band positions in an electrophoretic gel separation is disclosed in U.S. Pat. No. 5,096,557, which issued Mar. 17, 1992. At least one control is added directly to sample materials. The control with the sample materials are subjected to electrophoresis. Following electrophoretic separation, there is a comparison of the sample materials to the control.

### SUMMARY OF THE INVENTION

A first aspect of the invention relates to an electrophoretic separation system configured to determine a size of each of a plurality of sample polynucleotides. One embodiment of the system includes a number N¹ sample separation lanes, wherein each sample separation lane is configured to subject a number N^{S} respective sample polynucleotides and a respective internal standard polynucleotide (ISP) to electrophoresis, the N¹ sample separation lanes having an index k, where k=1, 2, 3, . . . N¹, and the N^{S} sample polynucleotides of the kth separation lane having an index j, where j=1, 2, 3, . . . N^{S}, a ladder separation lane for subjecting a polynucleotide ladder to electrophoresis, the polynucleotide ladder comprising at least two polynucleotide ladder members (PLMs), and a processor configured to determine migration coordinates of (1) the ISP and Ns sample polynucleotides of each of the N¹ sample separation lanes and (2) at least two of the PLMs, and wherein the processor is further configured to transform the migration coordinates of the N^{S} sample polynucleotides of each separation lane from a migration dimension of their respective separation lane to a migration dimension of the polynucleotide ladder and determine the size of the N^{S} sample polynucleotides based on (1) the respective transformed migration coordinates thereof and (2) migration coordinates of at least two PLMs.

The migration coordinates may preferably be a migration time, migration distance, or a migration coordinate determined from a combination of migration time and migration distance.

In a preferred embodiment, the processor is configured to determine a transformed migration coordinate M_{I}_{ₖ}^{T} of the ISP in the kth separation lane and then transform the migration coordinates of the sample polynucleotides of the kth separation lane based on M_{I}_{ₖ}^{T}. For example, M_{I}_{ₖ}^{T} may be determined by a function:

M_{I}_{ₖ}^{T}=f=(Δₘ,Δ_{S(IL)})

where (1) Δ_{S(IL)} is a size difference between the ISP subjected to electrophoresis in the kth separation lane and at least one PLM, and (2) Δm is a rate of change of the migration coordinate of the PLMs as a function of a size of the PLMs, wherein the Δm is determined from at least two of the PLMs. Thus, the processor may determine the transformed migration coordinate M_{I}_{ₖ}^{T} by:

M_{I}_{ₖ}^{T}=M_{L}±Δₘ×(S_{I}_{ₖ}±S_{L}_{ᵢ})

where (1) M_{L} is a value determined from a migration coordinate of at least one of the PLMs, (2) S_{I}_{ₖ} is the size of the ISP of the kth separation lane, and (3) S_{L}_{ᵢ} is the size of the ith PLM.

In another embodiment, the processor is configured to determine the size S_{S}_{ⱼₖ} of the jth sample polynucleotide subjected to electrophoresis in the kth separation lane by a function:

where (1) M_{S}_{ⱼₖ}^{T} is the transformed migration coordinate of the jth sample polynucleotide of the kth separation lane and (2) Δ′ₘ is a rate of change of the migration coordinate of the PLMs as a function of a size of the PLMs, wherein the Δ′ₘ is determined from at least two of the PLMs.

The processor may be configured to determine M_{I}_{ₖ}^{T} from a parameter obtained by fitting a function to the migration coordinates of a plurality of the PLMs. The function may have at least one of a quadratic term and an exponential term. The processor may be configured to determine the size of the jth sample polynucleotide subjected to electrophoresis in the kth separation lane based upon at least one parameter, such as a slope or other coefficient, obtained from the function.

In yet another embodiment, the processor is configured to determine a quantity of the jth sample polynucleotide subjected to electrophoresis in the kth separation lane based upon a detected fluorescence intensity of the ISP subjected to electrophoresis in the kth separation lane and a detected fluorescence intensity of the sample polynucleotide.

A preferred embodiment of the system comprises at least 96 separation lanes including the ladder separation lane.

Another aspect of the invention relates to a method for determining a size of a plurality of sample polynucleotides. The method comprises subjecting a plurality of mixtures each comprising (1) a number N^{S} sample polynucleotides and (2) an internal standard polynucleotide (ISP) to electrophoresis along a bore of each of a respective one of a number N¹ separation lanes, the N¹ separation lanes having an index k, where k=1, 2, 3, . . . N¹, and the N^{S} sample polynucleotides of the kth separation lane having an index j, where j =1, 2, 3, . . . N^{S}, subjecting a polynucleotide ladder to electrophoresis along a bore of a different separation lane, the polynucleotide ladder comprising at least two polynucleotide ladder members (PLMs), determining migration coordinates of the sample polynucleotides, the standard polynucleotides and at least two of the PLMs, transforming the migration coordinates of the N^{S} sample polynucleotides of each separation lane from a migration dimension of their respective separation lane to a migration dimension of the polynucleotide ladder, and determining the sizes of the sample polynucleotides based on at least (1) the respective transformed migration coordinates thereof and (2) migration coordinates of the PLMs.

The migration coordinates of the sample polynucleotides in the kth separation lane are determined on the basis of a transformed migration coordinate M_{I}_{ₖ}^{T} of the ISP of the kth separation lane. For example, M_{I}_{ₖ}^{T} may be determined by a function:

M_{I}_{ₖ}^{T}=f(Δₘ,Δ_{S(IL)})

where (1) Δ_{S(IL)} is a size difference between the ISP subjected to electrophoresis in the kth separation lane and at least one PLM, and (2) Δm is a rate of change of the migration coordinate of the PLMs as a function of a size of the PLMs, wherein the Am is determined from at least two of the PLMs. The transformed migration coordinate M_{I}_{ₖ}^{T} may be determined by:

M_{I}_{ₖ}^{T}=M_{L}±Δₘ×(S_{I}_{ₖ}±S_{L}_{ᵢ})

where (1) M_{L} is a value determined from a migration coordinate of at least one of the PLMs, (2) S_{I}_{ₖ} is the size of the ISP of the kth separation lane, and (3) S_{L}_{ᵢ} is the size of the ith PLM.

In one embodiment, the size S_{S}_{ⱼₖ} of the jth sample polynucleotide subjected to electrophoresis in the kth separation lane by:

S_{S}_{ⱼₖ}=f(Δ′ₘ,M_{S}_{ⱼₖ}^{T})

where M_{S}_{ⱼₖ}^{T} is the transformed migration coordinate of the jth sample polynucleotide of the kth separation lane and Δ′m is a rate of change of the PLMs as a function of their sizes.

M_{I}_{ₖ}^{T} may be determined by fitting a function to the migration coordinates of a plurality of members of the polynucleotide ladder. The function may include, for example, at least one of a quadratic term and an exponential term. The size of the jth sample polynucleotide subjected to electrophoresis in the kth separation lane may be determined based upon at least one parameter obtained from the function.

The sample polynucleotides may be amplicons resulting from amplification of a parent polynucleotide.

A quantity of the jth sample polynucleotide subjected to electrophoresis in the kth separation lane may be determined based upon a detected fluorescence intensity of the ISP subjected to electrophoresis in the kth separation lane and a detected fluorescence intensity of the jth sample polynucleotide in the kth separation lane.

Yet another aspect of the present invention relates to a computer-readable medium comprising executable software code, the code for processing electrophoresis data to determine a size of at least one sample polynucleotide, the electrophoresis data comprising (1) a first subset of data comprising peaks indicative of a separation of (a) at least one polynucleotide and (b) at least one internal standard along a first sample separation lane and (2) a second subset of data comprising a plurality of peaks indicative of a separation of members of a molecular ladder along a ladder separation lane, the computer-readable medium comprising code to determine a migration coordinate of at least one peak corresponding to the presence of an internal standard subjected to electrophoresis along the sample separation lane, code to determine a migration coordinate of at least one peak corresponding to the presence of the sample polynucleotide subjected to electrophoresis along the sample separation lane, code to determine migration coordinates of at least two members of the molecular ladder subjected to electrophoresis along the ladder separation lane, code to transform the migration coordinate of the sample polynucleotide from a migration dimension of the sample separation lane to a migration dimension of the ladder separation lane, and code to determine the size of the sample polynucleotide based on at least (1) the transformed migration coordinate of the peak of the sample polynucleotide and (2) migration coordinates of peaks of at least two of members of the molecular ladder.

### BRIEF DESCRIPTION OF THE DRAWINGS

The present invention is described below in reference to the Drawings in which:

FIG. 1 shows a flow chart of an embodiment of the method of the present invention;

FIG. 2 shows an electrophoretic separation system of the present invention;

FIG. 3 shows exemplary electrophoresis data including a peak;

FIG. 4 shows a plot of migration time v. size in base pairs obtained from electrophoresis of a 100 bp molecular ladder;

FIG. 5a shows exemplary electrophoresis data for electrophoresis of a molecular ladder and for electrophoresis of sample-internal standard mixtures;

FIG. 5b shows the electrophoresis data of FIG. 5a after transformation according to the present invention;

FIG. 6 shows a flow chart of a method for transforming electrophoresis data in accordance with the present invention;

FIG. 7 shows a flow chart of steps executed by exemplary computer code in accordance with the present invention; and

FIGS. 8a-8g show electrophoresis data obtained from capillary array electrophoresis of a molecular ladder and 6 sample-internal standard mixtures.

### DETAILED DESCRIPTION

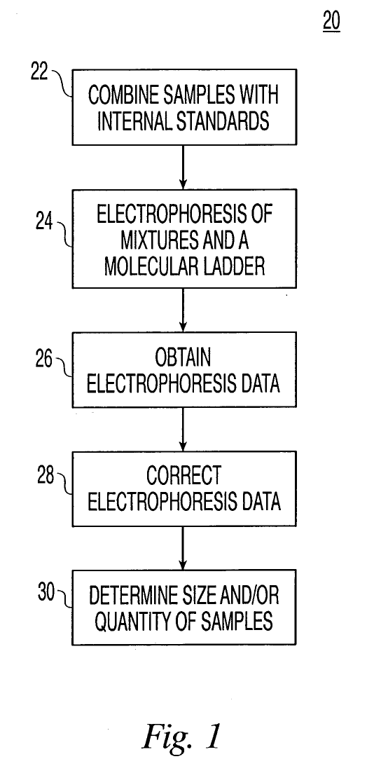

Referring to a flow chart 20 of FIG. 1, one embodiment of the present invention relates generally to determining the size of nucleotide containing compounds (NCC's) present in a sample. Alternatively, or in combination with size determination, the NCC's may be quantified to thereby determine the amount of NCC present. As used herein, the term “sample” refers to an NCC to be subjected to size or quantitation analysis in accordance with the present invention. Exemplary NCC's include polynucleotides such as single and double stranded DNA.

A preferred embodiment of the invention includes combining one or more sample NCC's with at least one internal standard to prepare 22 a plurality of mixtures. The internal standard may be NCC having a known size. The sample NCC's may be amplicons obtained by the amplification of one or more parent NCC's. As used herein, the term “parent NCC” refers to an NCC, such as DNA, to be amplified by a process such as polymerase chain reaction (PCR), which process is known in the art. It should be understood, however, that the present invention does not require that the sample NCC's be obtained by a process including an amplification step.

The mixtures of the sample NCC's and internal standards are subjected to electrophoresis 24 along various separation lanes. A molecular ladder is subjected to electrophoresis along a different separation lane. Electrophoresis data 26 indicative of the presence of the components of the mixtures and molecular ladder are obtained from the electrophoresis. The electrophoresis data are corrected 28 for mobility differences or quantitation differences between the separation lanes.

The corrected data may be used 30 to more accurately and precisely determine the size and/or quantity of sample NCC's present in the mixtures. For example, the size of a polynucleotide may be determined with a relative standard deviation of about 5 percent or less. The accuracy of the size determination is about 1.0 percent or less.

Various aspects of the present invention are discussed below.

Molecular Ladders

A molecular ladder comprises a plurality of known member compounds differing in size or mass. Various members of the molecular ladder exhibit different migration times when subjected to electrophoresis in the presence of a sieving matrix. Exemplary ladders include 2, 5, 10 or even more member compounds. A preferred molecular ladder is a polynucleotide ladder comprising a plurality of member NCC's, which may be DNA fragments. The member NCC's may be referred to as “polynucleotide ladder members” or PLMs.

The molecular ladder preferably includes at least two members having respective sizes that straddle the sizes of the sample NCC's. For example, at least one molecular ladder member has about the same number of base pairs or fewer than the smallest sample NCC and at least one other molecular ladder member has about the same number of base pairs or more as the largest sample NCC. The molecular ladder may include members having sizes that straddle the size of the internal standard so that at least one molecular ladder member has about the same number of base pairs or fewer than the internal standard and at least one other member has about the same number of base pairs or more as the internal standard.

An example of a suitable molecular ladder is a 100 bp DNA ladder, which can be obtained from Promega (Madison, Wis.). The DNA ladder contains 11 polynucleotides ranging in size from 100 to 1,000 bp, plus an additional fragment at 1,500 bp. Another suitable ladder is the DNA Mass Ladder (Life Technologies Inc., Gaithersburg, Md.), which consists of an equimolar mixture of six DNA fragments of 100, 200, 400, 800, 1200 and 2000 bp.

Amplification

The polymerase chain reaction for amplifying an NCC is known in the art. Thus, at least one region of an NCC, such as a single or double stranded DNA molecule, may be amplified to provide an amplicon. According to the invention, the amplicons may be analyzed to determine their sizes and quantity.

Electrophoresis

Electrophoresis in accordance with the present invention may be performed using electrophoretic separation lanes known in the art. Exemplary separation lanes include the internal bores capillaries filed with a sieving matrix that causes NCC's of varying sizes to migrate with different mobilities. An exemplary instrument for performing capillary electrophoresis is disclosed in U.S. Pat. No. 6,027,627, which patent discloses an automated parallel electrophoretic system. The patent is incorporated by reference herein.

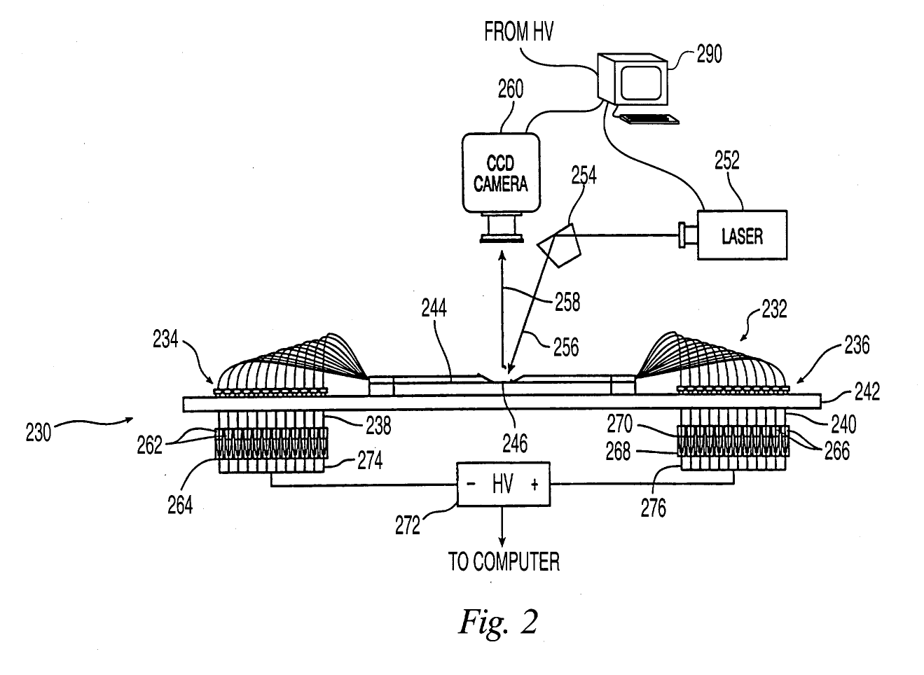

Referring to FIG. 2, an electrophoresis system 230 in accordance with the present invention includes a plurality of capillaries 232. A first array of ends 238 of the capillaries 232 may be spaced apart in substantially the same manner as the wells 262 of a microtitre tray 264. This allows one to simultaneously perform capillary electrophoresis on volumes of material present in each of the wells 262 of the tray 264. Thus, the molecular ladder and the sample NCC internal standard mixtures may be placed in various wells of the tray. The array of capillary ends 238 are placed in contact with the volumes of material in the wells 262. Upon the brief application of a current through the capillaries, an amount of the material is drawn into the respective capillaries of the array. The current is provided by a source 272 of high-voltage (HV) electricity. The array of capillary ends 238 is placed in contact with a solution of buffer. Upon the renewed application of an electric field to the capillaries 232, compounds previously drawn into the capillaries 232 from the wells 262 migrate toward a detection zone 246.

The detection zone 246 is spaced apart along a separation axis from the array of capillary ends 238. A detection system determines the presence of compounds in the detection zone. Exemplary detection systems include a light source 252 and a detector 260. Compounds present in the detection zone are irradiated with light 256 from the light source 252. A beam steering element 254 may be used to direct light 256 toward the detection zone. The irradiated compounds or fluorophores associated with the compounds may emit fluorescence 258. The irradiated compounds may also attenuate the irradiating light 256. The attenuated light or fluorescence is detected by the detector 260, which converts the light into a detector signal indicative of the presence or absence of the compounds. As understood in the art, the optical configuration used in an attenuation mode may be different from that shown in FIG. 2.

A computer 290 includes a computer-readable medium comprising code that when executed receives the detector signal from detector 260 and process the data in accordance with the invention. The code is discussed elsewhere herein.

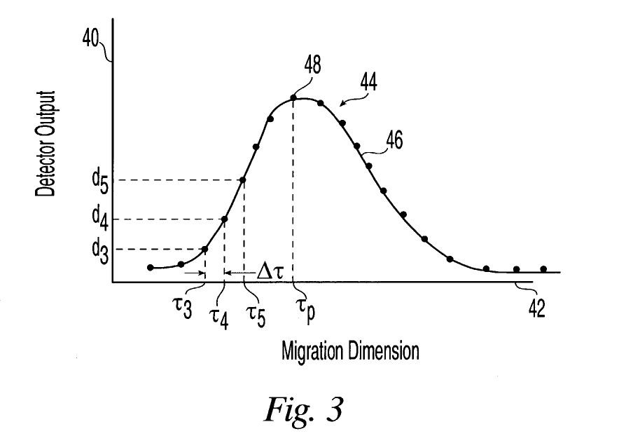

Referring to FIG. 3, electrophoresis data from a given separation lane include a detector coordinate dₑ and a migration coordinate mₑ. The detector coordinate dₑ is representative of the detector signal corresponding to the e^{th} position along a migration dimension 42. For example, detector coordinates d₃, d₄, and d₅ correspond to the detector signal obtained at respective migration times t₃, t₄, and t₅.

In general, the detector signal output is indicative of fluorescence intensity or absorbance. It should be understood, however, that values along the detector dimension need not be indicative of the amount of compound present in the detection zone. For example, the values along the detector dimension may represent only the presence or absence of a detectable compound without conveying further information about the amount of compound present.

The migration coordinate mₑ of an electrophoresis data point is indicative of the position along the migration dimension 42. The migration coordinate mₑ may be expressed in, for example, units of time, distance, or combination thereof. When the migration dimension 42 are expressed in units of time, the migration position of the e^{th} data point is the time τₑ at which the eth data point was acquired by the detector. Successive points τₑ and τₑ₊₁ are spaced apart by the data acquisition time Δτ, which is typically between about 0.1 and 2 seconds but may be more or less.

The migration coordinate mₑ of an electrophoresis data point may also be expressed in terms of distance along the migration dimension. One may acquire distance data directly or convert time data to distance data. Electrophoresis time data can be converted to distance data by, for example, plotting the data and then measuring the distances between a reference point and various peaks present in the data. To acquire distance data directly, a separation may be allowed to proceed for a total time T at which time further migration is stopped, such as by turning off the electric field applied to the separation lane. Subsequently, a detector is used to determine the presence or absence of compounds as a function of distance along the separation dimensions. This may be accomplished by scanning the length of a capillary through a light beam, which defines a detection zone. The detector determines the attenuation of the light beam by compounds present in the detection zone or measures fluorescence from these compounds. Although sample compounds and a molecular ladder may be subjected to simultaneous electrophoresis in the various lanes of the capillary electrophoresis instrument, simultaneous electrophoresis is not required.

Each electrophoresis data point, for example, (dₑ,τₑ), may be referred to as a frame. The frame number, Fₑ, is indicative of the coordinate of the eth frame along the migration dimension and is given by Fₑ=τₑ/Δτ. Thus, one can readily convert between migration time and frame number so that the location of the eth data point dₑ along the migration dimension may be expressed in terms of the migration time τₑ or the frame number Fₑ.

A portion of the data points (dₑ,mₑ) may generally define a peak 44, which is indicative of the presence of a compound, such as a sample. As understood in the art, the migration coordinate of a peak, such as the migration time τₚ of the peak, may be determined by fitting the peak to a peak-shape model 46 and determining the peak migration position from the parameters of the fitted peak. Alternatively, one may simply determine a peak migration time from a peak maximum 48 of the observed migration time data.

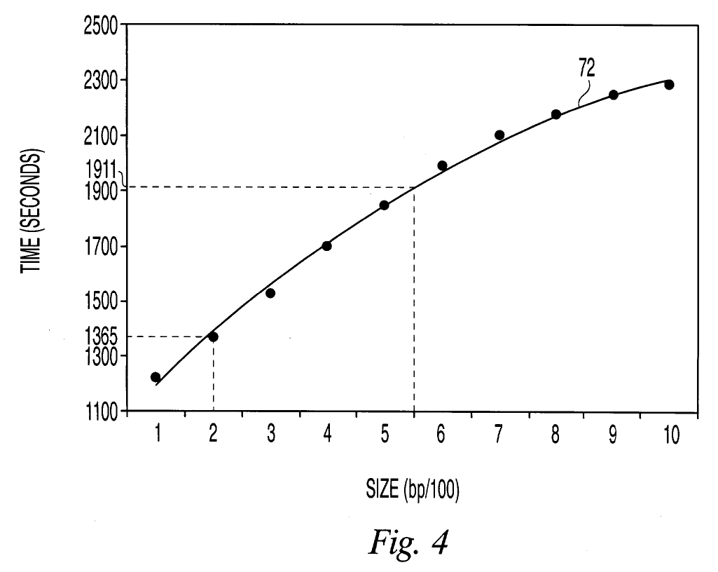

Referring to FIG. 4, the migration times (in seconds s) of members of a molecular ladder are plotted as a function of the size (in base pairs bp) of each member. For example, the migration time of the 200 base pair member of the molecular ladder is 1365 seconds. The electrophoresis data used to prepare the plot shown in FIG. 4 were acquired at a frame spacing Δτ of 0.99 seconds. Thus, for example, the 200 base pair member appears at frame number 1379.

A curve 72 represents a least squares fit of a second order polynomial τ=a+b·bp+c·bp² to the data of FIG. 4. The best-fit parameters were a=985.6 s, b=2.14 s/bp, and c=−8.3×10⁻⁴ s/bp². As shown by curve 72, the rate of change of the migration time with respect to the sizes of the members of the ladder changes along curve 72. Thus, the migration time as a function of ladder member size is generally non-linear over ranges of about 500 base pairs or more. Over smaller ranges, however, the migration time data may be better approximated by a linear function. Corresponding behavior would be observed if the data of FIG. 4 were plotted as a function of other migration dimensions such as distance or electrokinetic mobility.

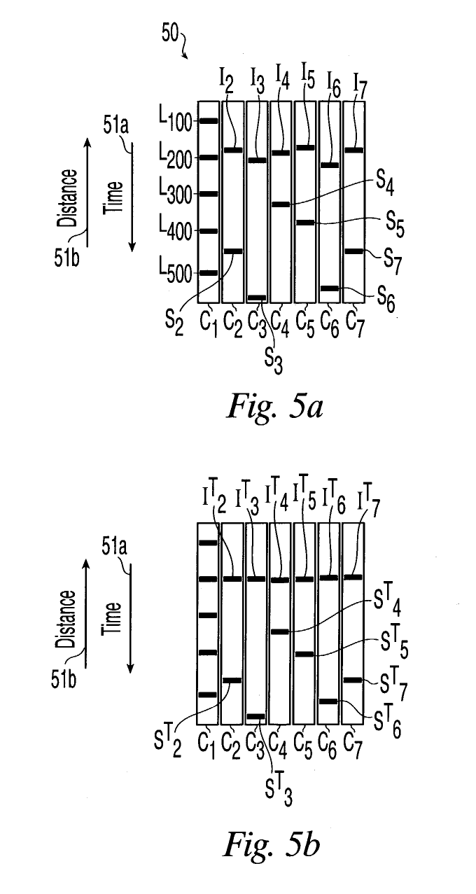

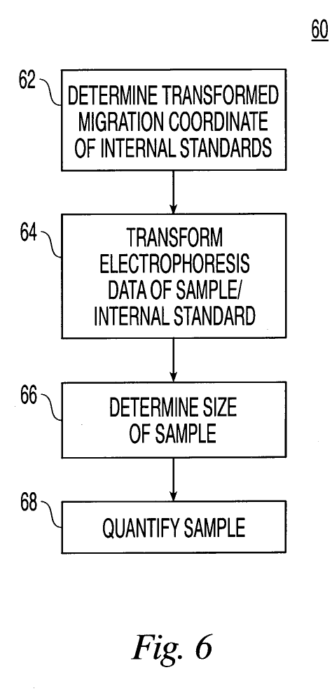

Referring to FIGS. 5a and 5b, respectively, untransformed, electrophoresis data and transformed electrophoresis data are shown in schematic form. Peaks designated as Lᵢ, where i is the length of the member in base pairs, are indicative of the presence of members of a 100 base pair molecular ladder. For example, peak L300 indicates the presence of a 300 bp member of the molecular ladder. The peaks Lᵢ are representative of peaks observed from the separation of the 100 base pair molecular ladder along a separation lane C₁.

Peaks respectively designated Sₖ and Iₖ, where k corresponds to a separation lane index, are indicative of the presence of various sample NCC's and internal standards. Thus, for example, peaks S₂ and I₂ respectively correspond to the presence of a sample and internal standard subjected to simultaneous electrophoresis along a single separation lane C₂. The migration coordinates of the various peaks may be expressed along a migration time dimension 51a, which corresponds to the time or frame number. The migration coordinates of the various peaks may also be expressed along a migration distance dimension 51b, as discussed above. It should be noted that more than one sample and internal standard may be simultaneously separated along each separation lane.

Although each of the internal standards present in separation lanes C₂-C₇ have the same size, 200 bp, mobility variations between the different separation lanes cause the internal standards to appear at different migration coordinates along the separation dimensions of the different separation lanes. Thus, the peaks Iₖ do not line up at the same time along the migration time dimension 51a. Additionally, the peaks Iₖ do not all line up with molecular ladder peak L₂₀₀, which has the same size as the internal standards. These variations show that the size of a sample present in one of separation lanes C₂-C₇ cannot be predicted accurately and precisely by a method that considers only (1) the migration coordinate of a sample peak subjected to electrophoresis in one of separation lanes C₂-C₇ and (2) the migration coordinate of molecular ladder peaks subjected to electrophoresis in separation lane C₁.

In accordance with one embodiment of the present invention, the precision and accuracy of size prediction is improved by correcting the migration time of a sample peak by a method that considers the migration coordinate of an internal standard subjected to electrophoresis in the presence of the sample compound in a first separation lane and the migration coordinates of molecular ladder peaks subjected to electrophoresis in a second, different separation lane. As discussed below, the present invention provides a method for correcting mobility variations in electrophoresis data acquired from various separation lanes to allow more accurate and precise prediction of the size and quantity of NCC's present in the separation lanes.

Data Analysis

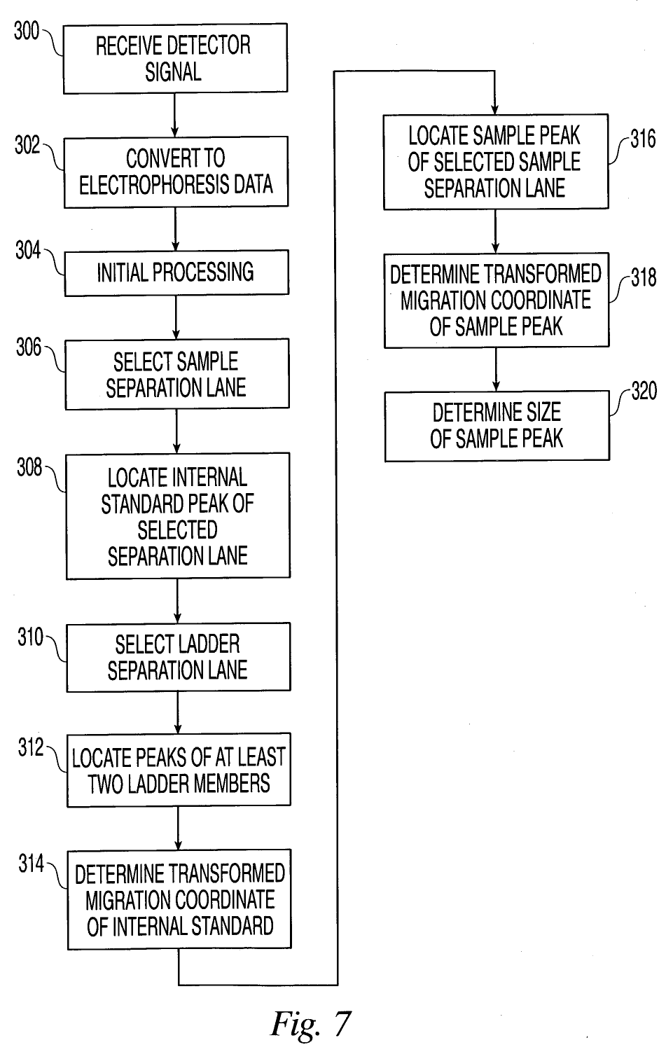

Referring to FIG. 6, a flow chart 60 includes steps for processing electrophoresis data in accordance with the invention. The discussion of flow chart 60 and steps therein refer to the analysis of electrophoresis data obtained from the separation of (1) at least one sample-internal standard mixture along a first separation lane; and (2) at least one molecular ladder along a second, different separation lane.

A transformed coordinate determining step 62 includes determining a transformed migration coordinate of the respective internal standard of each sample-internal standard mixture. An aligning step 64 uses the transformed migration coordinates to transform the sample-internal standard electrophoresis data with respect to the molecular ladder. A determine size step 66 includes using the transformed migration coordinates determined in step 64 to determine sizes of various samples of the sample-internal standard mixtures. The sample size may be determined in base pairs. A quantitation step 68 includes determining a concentration of the fragments underlying peaks present in the internal standard electrophoresis data.

Transformed Coordinate Determining Step 62

The alignment determining step 62 includes transforming a migration coordinate of each internal standard peak to a migration dimension of the molecular ladder. Exemplary transformed migration coordinates of the internal standard subjected to electrophoresis along the kth separation lane include a transformed migration time τ_{I}_{ₖ}^{T} and a transformed frame number F_{I}_{ₖ}^{T}.

A transformed migration coordinate of an internal standard is preferably determined using a migration coordinate rate of change, Δm. The Δm is the rate of change of the migration coordinate of members of the molecular ladder as a function of their sizes. A rate of change may be determined from the molecular ladder peaks using Eq. 1:

$${Δ}_{m} = \frac{( {M}_{{L}_{i}} - {M}_{{L}_{n}} )}{( {S}_{{L}_{i}} - {S}_{{L}_{n}} )} \qquad ( 1 )$$

where M_{Li} is the migration coordinate of the ith molecular ladder peak, M_{Ln} is the migration coordinate of the nth molecular ladder peak, S_{Li} is the size in base pairs of the compound underlying the ith molecular ladder peak, and S_{Ln} is the size in base pairs of the compound underlying the nth molecular ladder peak.

It is preferred, but not essential, that the sizes of the ith and nth members of the molecular ladder straddle the size of the internal standard. For example, the ith molecular ladder compound may have at least about as many bp or more as the internal standard and the nth molecular ladder compound may have about as few or fewer bp than internal standard.

The ith and nth molecular ladder compounds may be successively sized members of the molecular ladder so that the ith and nth molecular ladder peaks are adjacent to one another in electrophoresis data of the molecular ladder. It is not required, however, that the ith and nth peaks be successively sized. Additionally, either of the ith and nth peaks may have the larger size and, consequently, the longer migration time.

An exemplary Δm is a slope, Δτ, of migration time-base pair data obtained from the molecular ladder. A migration time slope Δτ may be determined by:

$${Δ}_{τ} = \frac{( {τ}_{{L}_{i}} - {τ}_{{L}_{n}} )}{( {S}_{{L}_{i}} - {S}_{{L}_{n}} )} \qquad ( 2 )$$

where τ_{Li} is the migration time of the ith molecular ladder peak and τ_{Ln} is the migration time of the nth molecular ladder peak. For example, the slope determined from the L200 and L300 peaks of the data of FIG. 4 is about 1.6 seconds per base pair.

A transformed migration coordinate M_{I}_{ₖ}^{T} is determined for each internal standard of the kth separation lane. The transformation may be a function of (1) the migration coordinate rate of change, Δm and (2) a size difference Δ_{S(IL)} between the internal standard and at least one member of the molecular ladder. For example, M_{I}_{ₖ}^{T} may be determined by:

M_{I}_{ₖ}^{T}=M_{L}±Δₘ×(S_{I}_{ₖ}±S_{Li}) (3)

where S_{I}_{ₖ} is the size in base pairs of the internal standard of the kth capillary and M_{L} is a value determined from a migration coordinate of at least one of the members of the molecular ladder. For example, M_{L} may be equal to M_{Li} or a value determined therefrom.

The transformed migration coordinate of the internal standard may be determined in units of time, frame number, distance along the migration dimension, or mobility because one may convert between these coordinates, as discussed above.

According to the preceding discussion, the transformed migration coordinate of the internal standard may be determined on the basis of as few as two peaks of the molecular ladder. However, the transformed migration coordinate may also be determined from a greater number of molecular ladder peaks such as by fitting these peaks to a straight line or non-linear function of the migration coordinates of the peaks. Suitable non-linear functions include polynomial functions and exponential functions. For example, using the best fit parameters obtained from the polynomial fit to the data of FIG. 4, the transformed migration time τ_{I}^{T} of a 550 bp internal standard is predicted to be 985.6 s+550 bp×2.14 s/bp+(550 bp)²×−8.3×10⁻⁴ s/bp²=1911.5 seconds.

A Transforming Step 64

Alignment step 64 includes transforming the migration coordinates of the sample compounds from the migration dimensions of their respective separation lanes to the migration dimension of the molecular ladder separation lane. A transformed migration coordinate, M_{S}_{ⱼₖ}^{T}, of the jth sample compound subjected to electrophoresis in the kth separation lane is determined using the transformed migration coordinate M_{I}_{ₖ}^{T} of an internal standard of the kth separation lane. For example, M_{S}_{ⱼₖ}^{T} may be determined by correcting the untransformed migration coordinate of the sample by an amount determined from a difference between the transformed and untransformed migration coordinates of the internal standard:

M_{S}_{ⱼₖ}^{T}=M_{S}_{ⱼₖ}+M_{I}_{ₖ}^{T}−M_{I}_{ₖ} (4)

where M_{S}_{ⱼₖ} is the untransformed migration coordinate of the jth sample of the kth separation lane and M_{I}_{ₖ} is the untransformed migration coordinate of the internal standard of the kth separation lane. Thus, the untransformed migration coordinate of the jth sample in the kth separation lane is shifted by the same amount as the untransformed migration coordinate of the internal lane standard of the kth separation lane.

During the analysis of the electrophoresis data, the electrophoresis coordinates of each of the molecular ladder peaks may be offset by a constant amount. For example, where the electrophoresis coordinates are expressed in frames, an offset in units of frame number may be added to each peak in the ladder. The offset is preferably large enough so that, upon transformation of the migration coordinates of the sample peaks, none of the transformed sample peaks take on negative migration coordinates. Because the offset is constant, the rates of change of the migration coordinates of the molecular ladder are not modified by the offset.

Referring back to FIG. 5b, peaks of the internal standards of electrophoresis data have been transformed to the migration dimension of the molecular ladder. The transformed peaks, Iₖ^{T}, are aligned with the 200 bp member of the molecular ladder. Peaks of the various samples have also been transformed. The sizes of the samples underlying the various transformed sample peaks may be determined as discussed below.

A Determine Size Step 66

In accordance with size determination step 66, the size S_{Sjk} of the jth sample NCC subjected to electrophoresis along the kth separation lane is determined. The size determination may be based on a function of a migration rate of change Δ′ₘ and a transformed migration coordinate M_{S}_{ⱼₖ}^{T} of the sample compound. For example, the size determination may take place by a function:

S_{Sjk}=f(Δ′ₘ,M_{S}_{ⱼₖ}^{T}) (5)

where (1) M_{S}_{ⱼₖ}^{T} is the transformed migration coordinate of the jth sample polynucleotide of the kth separation lane and (2) Δ′ₘ is a rate of change of the migration coordinate of the PLMs as a function of a size of the PLMs, wherein the Δ′ₘ is determined from at least two of the PLMs. The Δ′ₘ may be determined from the same molecular ladder peaks used to determine the transformed migration time of the internal standard. Alternatively, other peaks of the molecular ladder may be used to determine a Δ′ₘ for the sample size determination. The symbols Δ′ₘ and Δ′ₘ are equivalent as used herein.

The designation Δ′ₘ is used rather than Am because the rate of change used to determine the size of the sample polynucleotides may be different from the rate of change used to determine the transformed migration coordinate of the internal standard. In any event, the size S_{Sjk} is preferably expressed in base pairs. For example, the size S_{Sjk} may be determined by:

$$\begin{matrix}{S}_{{S }_{jk}} = {S}_{{L}_{x}} + \frac{( {S}_{{L}_{x}} - {S}_{{L}_{z}} ) ( {M}_{{S}_{jk}}^{T} - {M}_{{L}_{x}} )}{( {M}_{{L}_{x}} - {M}_{{L}_{z}} )} \\ = {S}_{{L}_{x}} + \frac{( {M}_{{S}_{jk}}^{T} - {M}_{{L}_{x}} )}{{Δ}_{m}^{′}}\end{matrix} \qquad ( 6 )$$

where S_{Lx} and S_{Lz} are the respective sizes in base pairs of the xth and zth members of the molecular ladder and M_{Lx} and M_{Lz} are the respective migration coordinates of the xth and zth members of the molecular ladder. Either or both of the xth and zth molecular ladder members may be identical to the ith and nth molecular ladder members used to determine the transformed migration coordinate of the internal standard. The Δₘ determined from the xth and zth molecular ladder members may be different than the Δₘ determined from the ith and nth molecular ladder members and used to determine the transformed migration time of the internal standard. It is preferred, however, that the migration times of the xth and zth molecular ladder compounds straddle the migration time of the sample NCC.

A size determination may also be made using a linear or non-linear fit to a plurality of the molecular ladder peaks. For example, using the best fit parameters obtained from the polynomial fit to the molecular ladder migration time data of FIG. 4, the size of a sample having a transformed migration time of 1775 seconds is predicted, from an iterative solution to the equation 1775 s=a+b·bp+c·bp², to be about 446 base pairs.

Quantitation Step 68

Quantitation of a sample subjected to electrophoresis preferably involves a comparison of a fluorescence intensity of the sample to a fluorescence intensity of a known concentration of internal standard subjected to electrophoresis along the same separation lane. A comparison of fluorescence intensity may be performed by comparing a peak area of the sample with a peak area of the internal standard. Alternatively, one may compare intensities determined from a maximum intensity of the respective peaks. Co-injection and co-electrophoresis in the same separation lane ensure an equal loading of the sample and internal standard, thereby eliminating the effect of variations between separation lanes and successive electrophoresis runs.

Processor

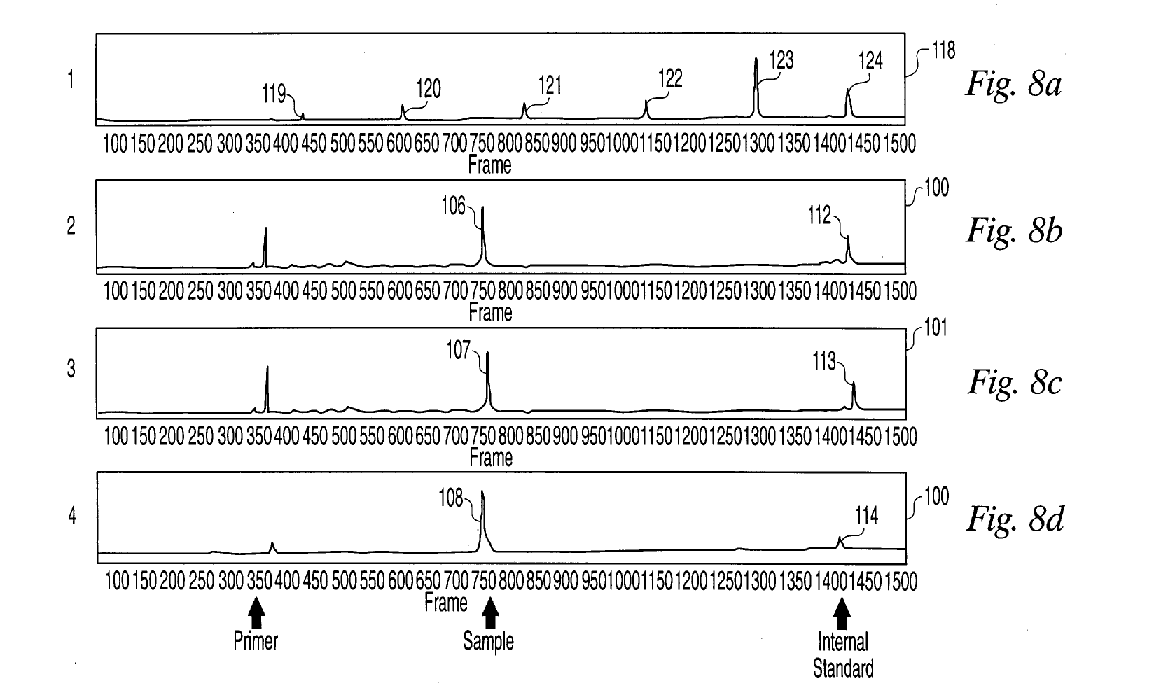

Referring to FIGS. 2 and 7, the electrophoresis system of the invention may include a computer or other processor configured to determine a size and/or quantity of one or more sample polynucleotides. The processor is typically implemented through a combination of hardware and executable software code. In the usual case, the processor includes a programmable computer, perhaps implemented as a reduced instruction set (RISC) computer, which handles only a handful of specific tasks. The computer is typically provided with at least one computer-readable medium, such as a PROM, flash, or other non-volatile memory to store firmware and executable software code, and will usually also have an associated RAM or other volatile memory to provide work space for data and additional software. Various steps that may be carried out by the computer or other processor in response to code of the computer-readable medium are discussed below in reference to the flow chart of FIG. 7.

Receive Detector Signal

The computer or processor is configured to receive 300 a detector signal. Because the computer may be either local to the electrophoresis instrument or remote therefrom, the computer may receive the detector signal through, for example, a hardwired connection, wireless connection, a network, a storage medium such as a disk, or combination thereof.

The code of the computer readable medium may, optionally, include code configured to convert 302 the detector signal to electrophoresis data. Because the detector may output a detector signal in the form of electrophoresis data including a detector coordinate dₑ and a migration coordinate me as discussed above in reference to FIG. 3, a conversion step may not be necessary.

Initial Conditioning

In certain situations, the raw data must be subjected to initial conditioning 304, such as by data smoothing, baseline subtraction, or by using deconvolution techniques to identify overlapped peaks. Suitable data conditioning techniques, such as those discussed below, are disclosed in U.S. application Ser. No. 09/676,526, filed Oct. 2, 2000, titled Electrophoretic Analysis System Having in-situ Calibration, which application is hereby incorporated to the extent necessary to understand the present invention. The computer-readable medium includes code to perform such conditioning.

Smoothing can be accomplished by using, for example, a Savitzky-Golay convoluting filter to improve the signal to noise ratio. Optimal properties of the filter, such as the width and order, can be determined by a user of the present invention on the basis of the signal to noise ratio of the data and the widths of peaks in the data.

Baseline subtraction can be performed to eliminate baseline drift. Typically, minima are identified in successive local sections of data, e.g., every 300 data points. Two or more minima in adjacent sections are connected, such as by a straight line or a polynomial fit to the minima. The values along the line connecting the minima are then subtracted from the intervening raw data. The new values after the baseline subtraction and smoothing are stored for further processing. The order of data smoothing and baseline subtraction can be reversed.

Overlapped peaks within the separations data can be identified and resolved using peak-fitting techniques. In most electrophoresis separations, the earlier-detected peaks are narrower than the later-detected, slower moving peaks. Within a given local section of data, however, peaks due to the presence of a single fragment have similar widths. Moreover, adjacent peaks rarely overlap exactly. Rather, the overlapped peaks a generally offset from one another. Accordingly, peaks due to the presence of multiple fragments tend to be wider than the single fragment peaks. Once a region of data containing overlapped peaks is identified, the underlying peaks can be resolved by fitting a model of the data to the observed data. Typically, the peak fitting model includes parameters that describe the amplitude, position, and width of each underlying peak.

Select Sample Separation Lane

The electrophoresis data may include data from each of a plurality of sample separation lanes. For example, the electrophoresis data may include a number N^{k} subsets of sample electrophoresis data each subset corresponding to the separation of a number N^{S} respective sample polynucleotides and a respective internal standard polynucleotide (ISP) along a respective one of a number N^{k} sample separation lanes. Thus, the code may configured to select 306, automatically or in response to user input, a subset of the electrophoresis data, the subset corresponding sample electrophoresis data of one or more of the sample separation lanes for further processing.

Locate Internal Standard Peak of Selected Sample Separation Lane

The code is configured to locate 308, either automatically or in response to user input, a peak corresponding to at least one internal standard of the selected sample separation lane. By locate, it is meant determine a migration coordinate, such as a migration time or distance, of the peak. For example the code may be configured to fit the peak to a peak shape model and determine the migration coordinate from the fitted parameters. Alternatively, locating the peak may include receiving a user input indicative of the migration coordinate of the peak. Locating the peak may also include finding the peak, such as by seeking intensity values of positions along the migration coordinate that exceed a threshold indicative of the presence of a peak.

Select Ladder Separation Lane

The electrophoresis data includes data from one or more ladder separation lanes. These data correspond to the separation of a molecular ladder along the ladder separation lane. The code may be configured to select 310, either automatically or in response to user input, a subset of the electrophoresis data corresponding to the separation of a particular molecular ladder.

Locate Peaks of at Least Two Ladder Members

The code may be configured to locate 312, either automatically or in response to user input, at least two peaks corresponding to members of the molecular ladder. For example, the software may be configured to find and locate ladder peaks that have migration times that straddle the migration time of the internal standard peak from a selected sample separation lane.

Determine Transformed Migration Coordinate of Internal Standard

The code may be configured to determine 314 the transformed migration coordinate of the internal standard peak of the selected sample separation lane. The transformation is performed in accordance transformation methods discussed herein.

Locate Sample Peak of Selected Sample Separation Lane

The code may be configured to locate 316, either automatically or in response to user input, at least one of the N^{S} peaks corresponding to the sample polynucleotides of the selected sample separation lane. The sample peak locating may also include finding the peak as discussed above.

Determine Transformed Migration Coordinate of Sample Peak

The code may be configured to determine 318 the transformed migration coordinate of the sample peak in accordance with the transformation methods discussed herein. This determination may include locating and, optionally, finding one or more additional peaks of the molecular ladder. For example, the code may include instructions to locate at least two molecular ladder member peaks, which straddle the sample peak.

Determine the Size of the Sample Peak

The code is configured to determine 320 the size of the sample peak in accordance with methods discussed herein.

### EXAMPLES

A standard PCR protocol was used to prepare two amplicons with respective sizes of 265 and 595 base pairs. A one hundredth dilution of each PCR product was made with 1× TE buffer (10 mM Tris-Cl, 1 mM EDTA) in 95 wells of a 96-well sample tray. A molecular ladder was added at a concentration of (0.13 μg/μl) to the remaining well of the tray. The molecular ladder was a 100 base pair DNA ladder obtained from Promega, Madison, Wis. This ladder contains 11 DNA fragments ranging in size from 100 to 1,000 bp, plus an additional fragment at 1,500 bp.

Capillary Electrophoresis

A Spectrumedix LLC model SCE9610 96 capillary array instrument was used to simultaneously separate the 95 sample-internal standard mixtures. A 96^{th} capillary of the array was used to separate the molecular ladder. The separation was performed using a gel matrix of 1% (w/v) 7-million molecular weight polyethylene oxide (PEO, Sigma-Aldrich, St. Louis, Mo.) dissolved in 1×TBE buffer that was reconstituted from commercial powder ReadyPack™ (Amresco, Solon, Ohio). The TBE buffer also served as the electrophoresis buffer.

Samples were injected into 96 capillaries of an electrophoresis system using a 10 second electrokinetic injection. Capillary electrophoresis was carried out at an electric field strength of 200 V/cm at a temperature of 35° C. for 40 min. Under these conditions, about 6 successive electrophoresis runs could be performed using the same matrix. The gel matrix could be replaced by flushing the capillaries for 25 minutes with a wash solution and refilling them with gel matrix.

Electrophoresis data were obtained by monitoring fluorescence of compounds migrating along the separation lanes. An intercalating dye, ethidium bromide, was added to the buffer system to enhance fluorescence detection of the DNA fragments. Fluorescence was excited using an argon-ion laser beam at 480 nm. Fluorescence emission was detected at 520 nm. The frame time Δτ was 0.99 seconds.

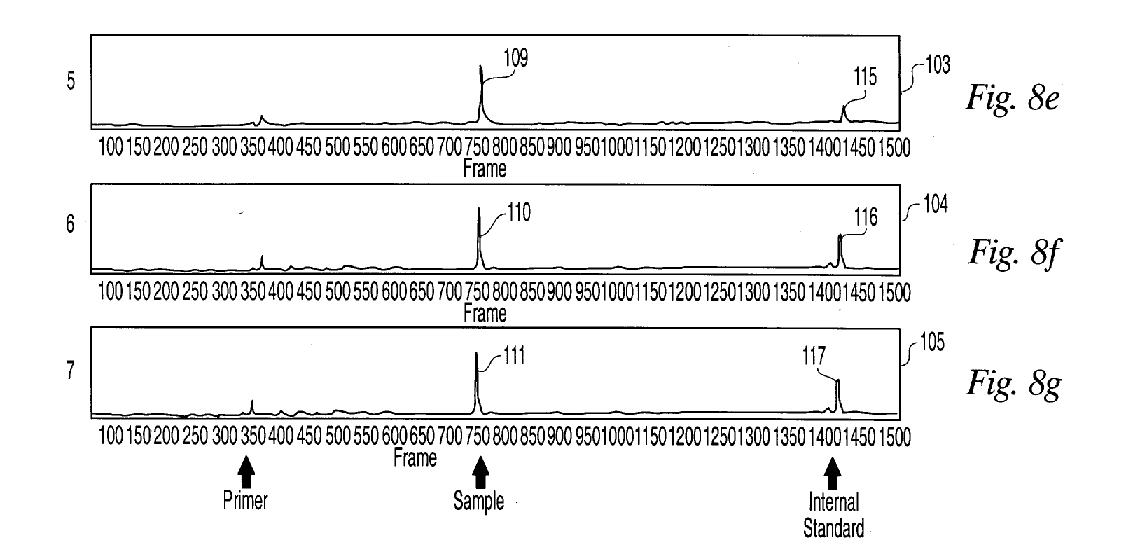

Referring to FIGS. 8a-8g, portions of the raw electrophoresis data obtained from 6 of the 95 capillaries in which the amplicons were subjected to electrophoresis and the capillary in which the molecular ladder was subjected to electrophoresis are shown. Electrophoresis data 118 of FIG. 8a include peaks 119-124 respectively indicative of the presence of the 100, 200, 300, 400, 500, and 600 base pair DNA fragments of the molecular ladder. Electrophoresis data 100-105 of FIGS. 8b-8g include respective peaks 106-111 indicative of the presence of the 265 base pair amplicon and respective peaks 112-117 indicative of the presence of the 595 base pair amplicon.

The electrophoresis data 100-105 and 118 are represented in units of frame number along the migration dimension, where each frame represents 0.99 seconds. In accordance with the present invention, a migration time was determined for peaks present in the electrophoresis data 100-105 and 118. Because of mobility variations among the various separation lanes, peaks 106-111 appear at various different frame numbers along the migration dimension even though the compounds underlying these peaks have the same size. Peaks 112-117 also appear at various different frame numbers.

Data Analysis

To demonstrate simultaneous size determination of a plurality of sample NCC's, the 595 base pair amplicon was treated as an internal standard and the 265 base pair amplicon was treated as a sample. An offset of 58 frames was added to each of the molecular ladder peaks to prevent the occurrence of transformed sample peaks having negative migration coordinates.

Referring to Table 1, transformed and untransformed migration times from peaks due to members of the molecular ladder and peaks due to the 265 bp fragment of capillaries 2-7 are shown. With the exception of the 595 bp fragment (internal standard) of capillary 3, the migration times of the 500 and 600 bp members of the molecular ladder completely straddle the migration times of the 595 bp amplicon. Using Eq. 2, a migration rate of change Δₘ of 1.66 s/bp was determined from the transformed 500 and 600 base pair molecular ladder peaks. Inserting the Δₘ into Eq. 3, a transformed migration time was determined for each peak corresponding to the 595 base pair amplicon:

M_{I}_{ₖ}^{T}=1458 s=1301 s+1.66 s/bp×(595 bp−500 bp)

Using the transformed internal standard migration times, the migration times of the 265 base pair fragments were transformed from the migration time dimension of their respective capillaries to the migration time dimension of the molecular ladder capillary. For example, using Eq. 4, the transformed migration time of the 265 bp fragment of the 7^{th} capillary was determined by:

M_{S}_{_{I,7}}^{T}=811 s=742 s+1459 s−1390 s

TABLE 1
Transformed and Untransformed Migration Times From Peaks Due
to Members of the Molecular Ladder and Peaks Due to the
265 and 595 bp Fragments of Capillaries 2-7.

| | Untransformed | Transformed |
|---|---|---|
| | Migration | Migration |
| PEAK | Coordinate (Frames) | Coordinate (Frames) |
| 200 bp Ladder Peak | 610 | 668 |
| 300 bp Ladder Peak | 827 | 885 |
| 500 bp Ladder Peak | 1243 | 1301 |
| 600 bp Ladder Peak | 1409 | 1467 |
| 265 bp fragment-capillary 2 | 754 | 807 |
| 265 bp fragment-capillary 3 | 760 | 804 |
| 265 bp fragment-capillary 4 | 743 | 819 |
| 265 bp fragment-capillary 5 | 753 | 808 |
| 265 bp fragment-capillary 6 | 748 | 812 |
| 265 bp fragment-capillary 7 | 742 | 811 |
| 595 bp fragment-capillary 2 | 1406 | 1459 |
| 595 bp fragment-capillary 3 | 1415 | 1459 |
| 595 bp fragment-capillary 4 | 1383 | 1459 |
| 595 bp fragment-capillary 5 | 1404 | 1459 |
| 595 bp fragment-capillary 6 | 1395 | 1459 |
| 595 bp fragment-capillary 7 | 1390 | 1459 |

The transformed migration times of the 265 bp amplicon and a migration rate of change determined from the 200 and 300 bp molecular ladder members were used to predict the size of this amplicon as if the size were unknown. For example, using equation 5, the size of the 265 bp amplicon of the 7^{th} capillary was predicted as:

$${S}_{{S}_{1 , 7}} = 265.4 bp = 200 bp + \frac{( 200 bp - 300 bp ) ( 810 s - 668 s )}{( 668 s - 885 s )}$$

The average predicted sizes for the 265 amplicons of capillaries 2-7 are shown below in Table 2. The average predicted size is 265±2.4 bp, which corresponds to a relative precision of better than %1.

TABLE 2
Predicted Sizes of the 265 bp amplicon.

| Capillary | Predicted Size of 265 bp Amplicon (bp) |
|---|---|
| 2 | 263.6 |
| 3 | 262.2 |
| 4 | 269.1 |
| 5 | 264.1 |
| 6 | 265.9 |
| 7 | 265.4 |

To further demonstrate the precision and accuracy with which the size of an NCC may be determined according to the present invention, 95 mixtures of the 265 and 595 bp amplicons were subjected to electrophoresis 12 times (n=12) in succession in 95 capillaries of the electrophoresis. The 100 bp DNA ladder was subjected to electrophoresis 12 times in a 96^{th} capillary. Using electrophoresis data acquired from the 12 sets of runs, each of the amplicons was successively treated as an internal standard to predict the size of the other amplicon, which was treated as the sample.

First, the size of the 265 bp amplicon was predicted as described above. Second, the 200 and 300 bp molecular ladder peaks were used to determine a transformed migration time for the 265 bp amplicon. A transformed migration time was then determined for the 595 bp amplicon based on the transformed migration time of the 265 bp amplicon. The size of the 595 bp amplicon was then predicted on the basis of the transformed migration time of this amplicon and the 500 and 600 bp molecular ladder peaks.

The results are shown in Table 3 and demonstrate that the present invention may be used to accurately and precisely determine the size of a polynucleotide.

TABLE 3
Precision and accuracy 01 sizing PCR products

| PCR product, | Average calculated | RSD, (%), |
|---|---|---|
| actual size (bp) | size (bp) | n = 12 |
| 265 | 266.6 | 1.504 |
| 595 | 592.3 | 0.946 |

Quantitation of PCR Amplicons

To test the SCE9610's performance on quantitation of DNA, the 100 bp DNA ladder was subjected to electrophoresis in all 96 capillaries. Various members of the molecular ladder treated as if they were co-injected samples of DNA. Areas underneath peaks corresponding to the members of the molecular ladder were determined by fitting the peaks to a peak shape function as known in the art.

Relative area ratios of various pairs of peaks were used to evaluate the reliability with which peak area could be estimated. The 500 bp member of the 100 bp DNA Ladder was used to calculate relative peak area ratios for each member of the ladder (Table 4). Only a small variation (relative standard deviaion (RSD), less than 10%) was detected on the data produced among 96 capillaries. A similar approach was then applied to estimate the relative ratios of different peak areas generated among various fragments of the DNA Mass Ladder (Table 5), in which relative concentration of each fragment is known. The estimated ratios are, generally, in agreement with the actual ratios calculated from the value provided by the manufacturer. The difference between estimated and actual ratios for some comparison is acceptable, considering the possibility that some variation originates from the molecular ladder. The RSD values were small among these measurements. These results suggest that repeatable analysis of peak area will provide a sensitive method for the determination of concentration of PCR products.

TABLE 4
Precision of comparing peak areas resulted from different DNA members
of the 100 bp Ladder. The values in the middle column illustrate the
relative ratio of peak area for each DNA member compared with
the 500-bp member.

| DNA fragments (bp) | Relative ratio of peak area | RSD (%) (n = 96) |
|---|---|---|
| 100 | 0.14 | 5.6 |
| 200 | 0.20 | 5.3 |
| 300 | 0.31 | 7.3 |
| 400 | 0.22 | 5.3 |
| 500 | 1 | — |
| 600 | 0.41 | 9.2 |
| 700 | 0.53 | 10.5 |
| 800 | 0.54 | 11 |
| 900 | 0.56 | 10.9 |
| 1000 | 0.48 | 8.9 |
| 1500 | 0.37 | 6.8 |

TABLE 5
Precision of estimating relative ratio of different peak areas generated
among various members of the DNA Mass Ladder

| Peak areas | Actual | Estimated | RSD (%) |
|---|---|---|---|
| compared | ratio* | ratio (avg) | n = 10 |
| 200 bp/400 bp | 0.5 | 0.63 | 9.36 |
| 400 bp/800 bp | 0.5 | 0.43 | 6.72 |
| 800 bp/1200 bp | 0.67 | 0.81 | 3.51 |
| 1200 bp/2000 bp | 0.6 | 0.6 | 5.31 |

*Calculated on the basis of the information provided by the manufacturer.

While the above invention has been described with reference to certain preferred embodiments, it should be kept in mind that the scope of the present invention is not limited to these. Thus, one skilled in the art may find variations of these preferred embodiments which, nevertheless, fall within the spirit of the present invention, whose scope is defined by the claims set forth below.

What is claimed is:

1. An electrophoretic separation system configured to determine a size of each of a plurality of sample polynucleotides, comprising:
a number N¹ sample separation lanes, wherein each sample separation lane is configured to subject a number N^{S} respective sample polynucleotides and a respective internal standard polynucleotide (ISP) to electrophoresis, the N¹ sample separation lanes having an index k, where k=1, 2, 3, . . . N¹, and the N^{S} sample polynucleotides of the kth separation lane having an index j, where j=1, 2, 3, . . . N^{S};
a ladder separation lane for subjecting a polynucleotide ladder to electrophoresis, the polynucleotide ladder comprising at least two polynucleotide ladder members (PLMs);
a processor configured to determine migration coordinates of (1) the ISP and N^{S} sample polynucleotides of each of the N¹ sample separation lanes and (2) at least two of the PLMs, and wherein the processor is further configured to:
transform the migration coordinates of the N^{S} sample polynucleotides of each separation lane from a migration dimension of their respective separation lane to a migration dimension of the polynucleotide ladder; and
determine the size of the N^{S} sample polynucleotides based on (1) the respective transformed migration coordinates thereof and (2) migration coordinates of at least two PLMs.

2. The system of claim 1, wherein each migration coordinate is a migration time.

3. The system of claim 1, wherein each migration coordinate is a migration distance.

4. The system of claim 1, wherein each migration coordinate is determined from a combination of migration time and migration distance.

5. The system of claim 1, wherein, the processor is configured to determine a transformed migration coordinate M_{I}_{ₖ}^{T} of the ISP in the kth separation lane and then transform the migration coordinates of the sample polynucleotides of the kth separation lane based on M_{I}_{ₖ}^{T}.

6. The system of claim 5, wherein M_{I}_{ₖ}^{T} is determined by a function:
M_{I}_{ₖ}^{T}=f(Δₘ,Δ_{S(IL)})
where (1) Δ_{S(IL)} is a size difference between the ISP subjected to electrophoresis in the kth separation lane and at least one PLM, and (2) Δm is a rate of change of the migration coordinate of the PLMs as a function of a size of the PLMs, wherein the Am is determined from at least two of the PLMs.

7. The system of claim 6, wherein the processor determines the transformed migration coordinate M_{I}_{ₖ}^{T} by:
M_{I}_{ₖ}^{T}=M_{L}±Δₘ×(S_{I}_{ₖ}±S_{L}_{ᵢ})
where (1) M_{L} is a value determined from a migration coordinate of at least one of the PLMs, (2) S_{I}_{ₖ} is the size of the ISP of the kth separation lane, and (3) S_{L}_{ᵢ} is the size of the ith PLM.

8. The system of claim 5, wherein the processor is configured to determine the size S_{S}_{ⱼₖ} of the jth sample polynucleotide subjected to electrophoresis in the kth separation lane by a function:
S_{S jk}=f(Δ′ₘ,M_{S}_{ⱼₖ}^{T})
where (1) M_{S}_{^{jk}}^{T} is the transformed migration coordinate of the jth sample polynucleotide of the kth separation lane and (2) Δ′ₘ is a rate of change of the migration coordinate of the PLMs as a function of a size of the PLMs, wherein the Δ′ₘ is determined from at least two of the PLMs.

9. The system of claim 5, wherein the processor is configured to determine M_{I}_{ₖ}^{T} from a parameter obtained by fitting a function to the migration coordinates of a plurality of the PLMs.

10. The system of claim 9, wherein the function has at least one of a quadratic term and an exponential term.

11. The system of claim 9, wherein the processor is configured to determine the size of the jth sample polynucleotide subjected to electrophoresis in the kth separation lane based upon at least one parameter obtained from the function.

12. The system of claim 1, wherein the processor is configured to determine a quantity of the jth sample polynucleotide subjected to electrophoresis in the kth separation lane based upon a detected fluorescence intensity of the ISP subjected to electrophoresis in the kth separation lane and a detected fluorescence intensity of the sample polynucleotide.

13. The system of claim 1, wherein the system comprises at least 96 separation lanes including the ladder separation lane.

14. A method for determining a size of a plurality of sample polynucleotides, comprising:
subjecting a plurality of mixtures each comprising (1) a number N^{S} sample polynucleotides and (2) an internal standard polynucleotide (ISP) to electrophoresis, at least one mixture being subjected to electrophoresis along a respective one of a number N¹ separation lanes, the N¹ separation lanes having an index k, where k=1, 2, 3, . . . N¹, and the N^{S} sample polynucleotides of the kth separation lane having an index j, where j=1, 2, 3, . . . N^{S};
subjecting a polynucleotide ladder to electrophoresis along a bore of a different separation lane, the polynucleotide ladder comprising at least two polynucleotide ladder members (PLMs);
determining migration coordinates of the sample polynucleotides, the standard polynucleotides and at least two of the PLMs;
transforming the migration coordinates of the N^{S} sample polynucleotides of each separation lane from a migration dimension of their respective separation lane to a migration dimension of the polynucleotide ladder; and
determining the sizes of the sample polynucleotides based on at least (1) the respective transformed migration coordinates thereof and (2) migration coordinates of the PLMs.

15. The method of claim 14, wherein each migration coordinate is a migration time.

16. The method of claim 14, wherein each migration coordinate is a migration distance.

17. The method of claim 14, wherein each migration coordinate is determined from a combination of migration time and migration distance.

18. The method of claim 14, wherein the migration coordinates of the sample polynucleotides in the kth separation lane are determined on the basis of a transformed migration coordinate M_{I}_{ₖ}^{T} of the ISP of the kth separation lane.

19. The method of claim 18, wherein M_{I}_{ₖ}^{T} is determined by a function:
M_{I}_{ₖ}^{T}=f(Δₘ,Δ_{S(IL)})
where (1) Δ_{S(IL)} is a size difference between the ISP subjected to electrophoresis in the kth separation lane and at least one PLM, and (2) Δm is a rate of change of the migration coordinate of the PLMs as a function of a size of the PLMs, wherein the Δm is determined from at least two of the PLMS.

20. The method of claim 19, comprising determining the transformed migration coordinate M_{I}_{ₖ}^{T} by:
M_{I}_{ₖ}^{T}=M_{L}±Δₘ×(S_{I}_{ₖ}±S_{L}_{ᵢ})
where (1) M_{L} is a value determined from a migration coordinate of at least one of the PLMs, (2) S_{I}_{ₖ} is the size of the ISP of the kth separation lane, and (3) S_{L}_{ᵢ} is the size of the ith PLM.

21. The method of claim 18, comprising determining the size S_{S}_{ⱼₖ} of the jth sample polynucleotide subjected to electrophoresis in the kth separation lane by:
S_{S}_{ⱼₖ}=f(Δ′ₘ,M_{S}_{ⱼₖ}^{T})
where M_{S}_{ⱼₖ}^{T} is the transformed migration coordinate of the jth sample polynucleotide of the kth separation lane and Δ′m is a rate of change of the PLMs as a function of their sizes.

22. The method of claim 18, wherein M_{I}_{ₖ}^{T} is determined by fitting a function to the migration coordinates of a plurality of members of the polynucleotide ladder.

23. The method of claim 22, wherein the function has at least one of a quadratic term and an exponential term.

24. The method of claim 23, wherein the size of the jth sample polynucleotide subjected to electrophoresis in the kth separation lane is determined based upon at least one parameter obtained from the function.

25. The method of claim 14, wherein the sample polynucleotides are amplicons resulting from amplification of a parent polynucleotide.

26. The method of claim 14, further comprising determining a quantity of the jth sample polynucleotide subjected to electrophoresis in the kth separation lane based upon a detected fluorescence intensity of the ISP subjected to electrophoresis in the kth separation lane and a detected fluorescence intensity of the jth sample polynucleotide in the kth separation lane.

27. A computer-readable medium comprising executable software code, the code for processing electrophoresis data to determine a size of at least one sample polynucleotide, the electrophoresis data comprising (1) a first subset of data comprising peaks indicative of a separation of (a) at least one polynucleotide and (b) at least one internal standard along a first sample separation lane and (2) a second subset of data comprising a plurality of peaks indicative of a separation of members of a molecular ladder along a ladder separation lane, the computer-readable medium comprising:
code to determine a migration coordinate of at least one peak corresponding to the presence of an internal standard subjected to electrophoresis along the sample separation lane;
code to determine a migration coordinate of at least one peak corresponding to the presence of the sample polynucleotide subjected to electrophoresis along the sample separation lane;
code to determine migration coordinates of at least two members of the molecular ladder subjected to electrophoresis along the ladder separation lane;
code to transform the migration coordinate of the sample polynucleotide from a migration dimension of the sample separation lane to a migration dimension of the ladder separation lane; and
code to determine the size of the sample polynucleotide based on at least (1) the transformed migration coordinate of the peak of the sample polynucleotide and (2) migration coordinates of peaks of at least two of members of the molecular ladder.
